(19) United States
(12) Patent Application Publication
(10) Pub. No.: US 2010/0137340 A1
(43) Pub. Date: Jun. 3, 2010

(54) FUSED PYRIMIDINONE COMPOUNDS AS MGLUR LIGANDS

(76) Inventors: Ralf Glatthar, Bad Sackingen (DE); Samuel Hintermann, Basel (CH); Ivan-Toma Vranesic, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/529,593

(22) PCT Filed: Mar. 3, 2008
(86) PCT No.: PCT/EP08/52564

§ 371 Date: Sep. 2, 2009

(30) Foreign Application Priority Data
Mar. 5, 2007 (GB) 0704230.2

(51) Int. Cl. A61K 31/519; C07D 487/04; A61P 1/00; A61P 13/00; A61P 25/16; A61P 25/28; A61P 25/00; A61P 25/30

(52) U.S. Cl. 514/261.1; 544/262; 544/254; 514/262.1

(57) ABSTRACT

The invention relates to compounds of Formula (I) wherein the substituents are as defined in claim 1; to compositions comprising said compounds and to their use as pharmaceutical agents.

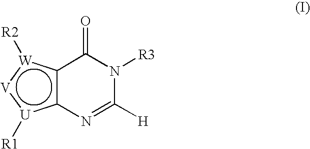

The present invention relates to novel heterocyclic derivatives, their preparation, their use as pharmaceuticals and pharmaceutical compositions containing them.

In a first aspect, the invention relates to a compound of formula (I) or a tautomeric form thereof

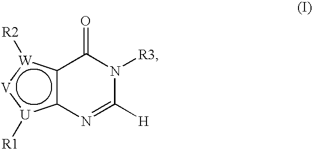

wherein
U represents C or N;
V represents CH, N, or O;
W represents C, N, or O;
R1 represents an optionally substituted aryl or an optionally substituted heteroaryl group;
R2 if present is selected from the group consisting of H, alkyl, aryl, hydroxy or alkoxy;
R3 is selected from the group consisting of optionally substituted cycloalkyl, optionally substituted heterocycloalkyl or 3-Cl-phenyl;
in free base or acid addition salt form,
with the proviso, that the compound of the formula

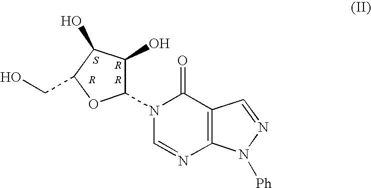

is excluded.

In the present specification, the following definitions shall apply, if no specific other definition is given:

“Alkyl” represents a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl.

“Alkandiyl” represents a straight-chain or branched-chain alkandiyl group bound by two different Carbon atoms to the molecule, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”.

“Alkenyl” represents a straight-chain or branched-chain alkenyl group, preferably C₂₋₆alkenyl, for example, vinyl, allyl, 1-propenyl, isopropenyl, 2-butenyl, 2-pentenyl, 2-hexenyl, etc. and preferably represents C₂₋₄ alkenyl.

“Alkendiyl” represents a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—.

“Alkynyl” represents a straight-chain or branched-chain alkynyl group, preferably C₂₋₆alkynyl, for example, ethenyl, propargyl, 1-propynyl, isopropenyl, 1-(2- or 3) butynyl, 1-(2- or 3) pentenyl, 1-(2- or 3) hexenyl, etc. preferably represents C₂₋₄alkynyl and particularly preferably represents ethynyl.

“Aryl” represents an aromatic hydrocarbon group, preferably a C₆₋₁₀ aromatic hydrocarbon group; for example phenyl, naphthyl, especially phenyl.

“Aralkyl” denotes an “Aryl” bound to an “Alkyl” (both as defined above) and represents, for example benzyl, α-methylbenzyl, 2-phenylethyl, α,α-dimethylbenzyl, especially benzyl.

“Heterocycle” represents a saturated, partly saturated or aromatic ring system containing at least one hetero atom. Preferably, heterocycles consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), Halogen, Nitro, Cyano, Alkyl, Alkandiyl, Alkenediyl, Alkoxy, Alkoxyalkyl, Alkoxycarbonyl, Alkoxycarbonylalkyl, Halogenalkyl, Aryl, Aryloxy, Arylalkyl. Examples of heterocyclic moieties are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazlolidine, isothiazole, istothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline and the like.

“Hetero atoms” are atoms other than Carbon and Hydrogen, preferably Nitrogen (N), Oxygen (O) or Sulfur (S).

“Halogen” represents Fluoro, Chloro, Bromo or lodo, preferably represents Fluoro, Chloro or Bromo and particularly preferably represents Chloro.

The term “cycloalkyl” refers to optionally substituted monocyclic, bicyclic or tricyclic hydrocarbon groups of 3-12 carbon atoms, each of which may contain one or more carbon to carbon double bonds, or the cycloalkyl may be substituted by one or more substituents, such as alkyl, halo, oxo, hydroxy, alkoxy, alkanoyl, acylamino, carbamoyl, alkylamino, dialkylamino, thiol, alkylthio, cyano, carboxy, alkoxycarbonyl, sulfonyl, sulfonamido, sulfamoyl, heterocyclyl and the like.

Exemplary monocyclic hydrocarbon groups include, but are not limited to, cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1 ]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

Some of the compounds of the formula (I) may exist in two or more tautomeric forms. The skilled person will recognise that the particular tautomeric form and/or the proportion of different tautomeric forms in which a compound of the invention exists may vary depending on the conditions to which the compound is subjected. All such tautomeric forms as well as mixtures thereof are part of the present invention.

Compounds of formula (I) exist in free or acid addition salt form. In this specification, unless otherwise indicated, language such as “compounds of formula (I)” is to be understood as embracing the compounds in any form, for example free base or acid addition salt form. Salts which are unsuitable for pharmaceutical uses but which can be employed, for example, for the isolation or purification of free compounds of formula (I), such as picrates or perchlorates, are also included. For therapeutic use, only pharmaceutically acceptable salts or free compounds are employed (where applicable in the form of pharmaceutical preparations), and are therefore preferred.

On account of the asymmetrical carbon atom(s) that may be present in the compounds of formula (I) and their salts, the compounds may exist in optically active form or in form of mixtures of optical isomers, e.g. in form of racemic mixtures or diastereomeric mixtures. All optical isomers and their mixtures, including the racemic mixtures, are part of the present invention.

Preferred substituents, preferred ranges of numerical values or preferred ranges of the radicals present in the formula (I) and the corresponding intermediate compounds are defined below.

U—V—W together prefreably represent: N—N—C; N—CH—N; C—N—N; N—N—N; C—N—O; or C—O—N.

R1 represents an optionally substituted aryl or an optionally substituted heteroaryl group, wherein the aryl or heteroaryl ring is preferably six-membered, in particular phenyl or a six-membered heteroaryl ring containing one heteroatom, in particular N. R1 may be an unsubstituted group. If substituted, the aryl or heteroaryl group which is represented by R1 carries preferably 1 to 3, in particular 1 substituent. Preferred substituents are halogen, in particular chloro, and lower alkyl, in particular methyl.

R2, if present, is preferably selected from the group consisting of H, lower alkyl, phenyl, hydroxy or lower alkoxy.

R3 is preferably selected from the group consisting of an optionally substituted C5-C8 monocyclic alkyl group, an optionally substituted bicyclic alkyl group containing 7 to 12 carbon atoms in the bicyclic moiety, an optionally substituted heterocyclic group dervivable from an optionally substituted C5-C8 monocyclic alkyl group or an optionally substituted bicyclic alkyl group containing 7 to 12 carbon atoms in the bicyclic moiety by replacing a CH group in one of the rings by a nitrogen atom and 3-chloro phenyl.

In particularly preferred compounds, R1-U is selected from the group consisting of

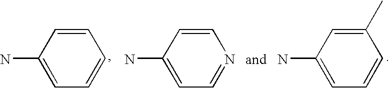

In particularly preferred compounds, V is N.

In particularly preferred compounds, R2-W is selected from the group consisting of CH and N.

In particularly preferred compounds, R3 is selected from the group consisting of cyclohexyl, cycloheptyl, cyclooctyl,

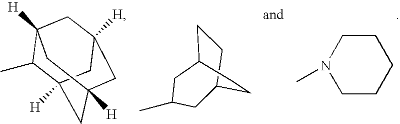

The abovementioned general or preferred radical definitions apply both to the end products of the formula (I) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formula (I) which contain a combination of the meanings mentioned above as being preferred.

Particular preference according to the invention is given to compounds of the formula (I) which contain a combination of the meanings listed above as being particularly preferred.

Specific example compounds are as follows:

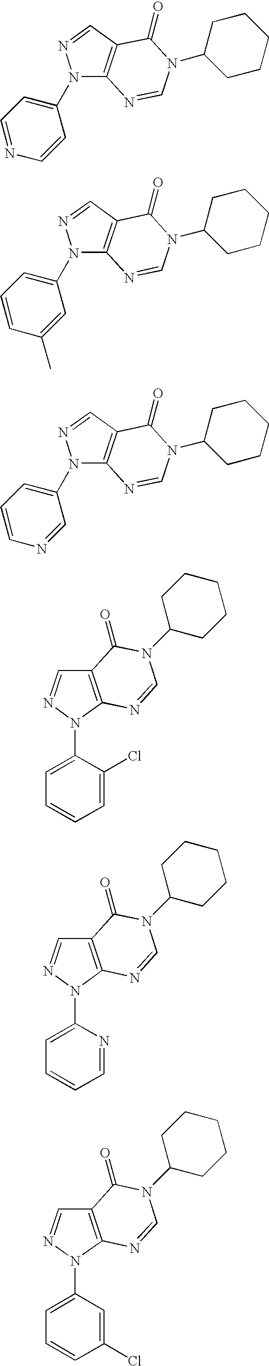

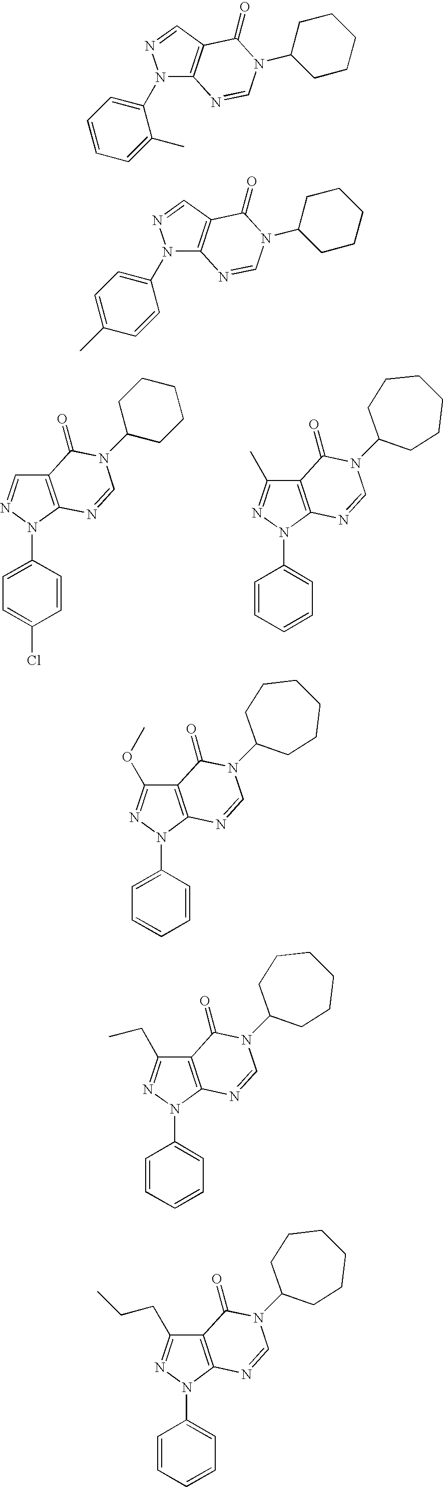

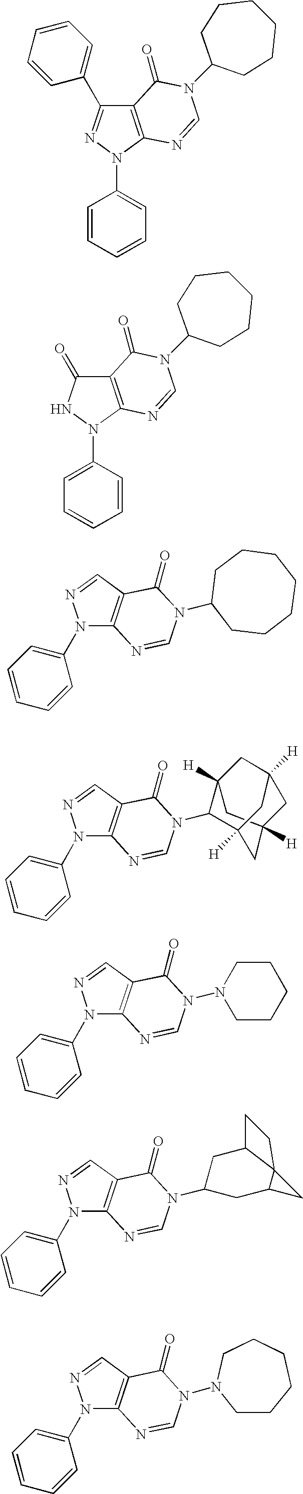

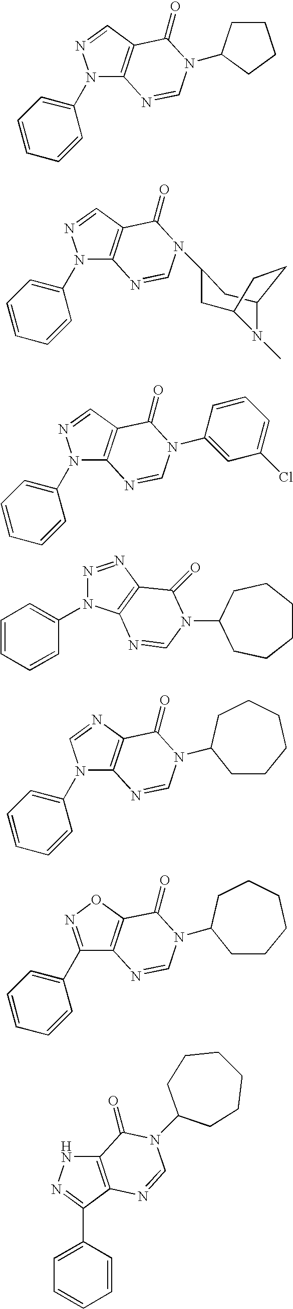

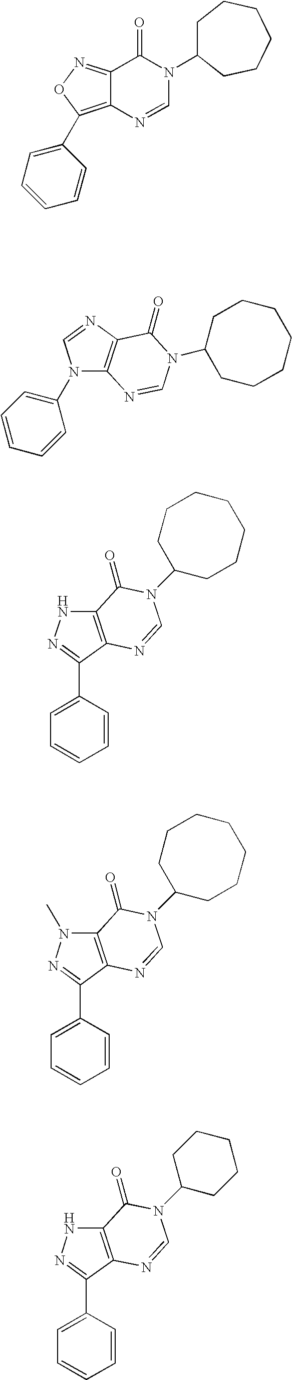

Some especially preferred example compounds are listed below:

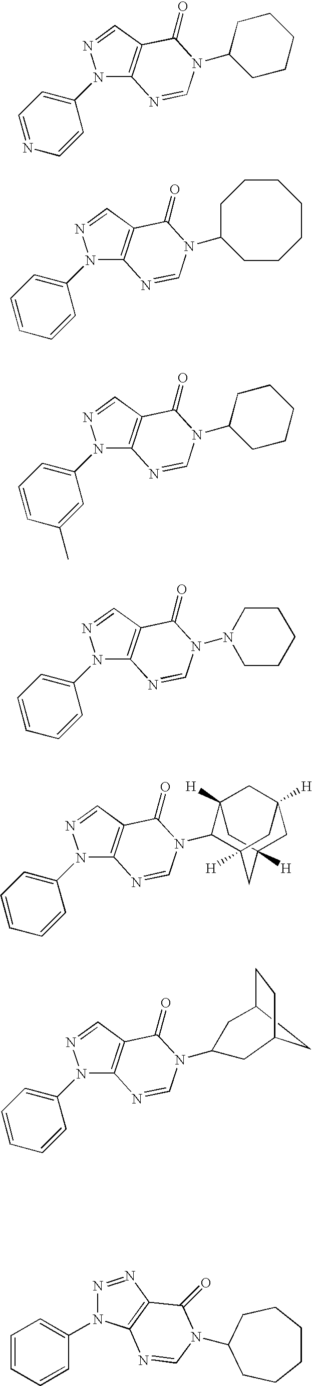

In a further aspect, the invention provides processes for the production of the compounds of formula (I) and their salts. It is noted that the processes depicted below are multistep processes. However, the individual steps as well as the intermediates formed are also part of the invention. The skilled person will recognize that the individual steps or intermediates are useful as such and can be combined in different sequences, for instance to provide alternative routes for the preparation of compounds according to the present invention

A first process which is useful for the preparation of compounds of the general formula (I) wherein U—V—W together represent N—N—C comprises the following steps:

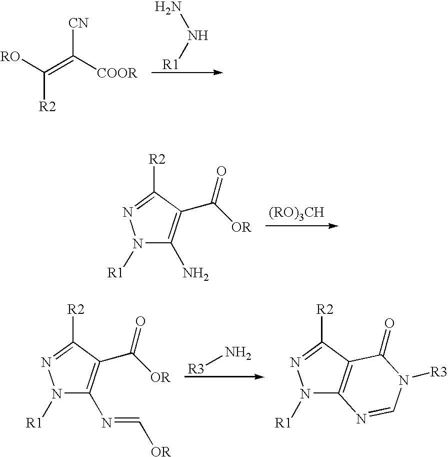

On each occurrence, R is an independently selected organic residue, preferably an alkyl group, in particular a lower alkyl group and most preferably an ethyl group. R1, R2 and R3 are defined as above.

Suitable reagents and reaction conditions can be determined by the skilled person. The first step of the above reaction is preferably carried out in a solvent, in particular ROH, wherein R is defined as above. The reaction is preferably carried out with heating. The second step is preferably carried out in the presence of an activating compound, such as p-TsOH. The reaction is preferably carried out with heating. The third step of the above reaction is preferably carried out in a solvent, in particular ROH, wherein R is defined as above. The reaction is preferably carried out with heating.

A particularly preferred embodiment of the first process is depicted below:

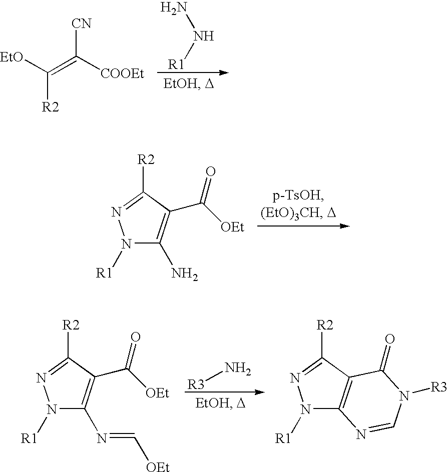

A second process which is useful for the preparation of compounds of the general formula (I) comprises the following steps:

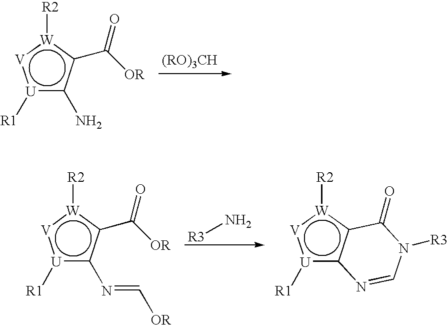

On each occurrence, R is an independently selected organic residue, preferably an alkyl group, in particular a lower alkyl group and most preferably an ethyl group. U, V and W as well as R1, R2 and R3 are defined as above.

Suitable reagents and reaction conditions can be determined by the skilled person. The first step is preferably carried out in the presence of an activating compound, such as p-TsOH. The reaction is preferably carried out with heating. The second step of the above reaction is preferably carried out in a solvent, in particular ROH, wherein R is defined as above. The reaction is preferably carried out with heating.

A particularly preferred embodiment of the second process is depicted below:

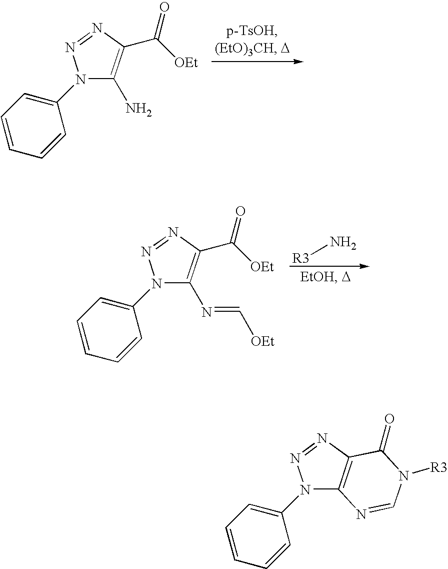

A third process which is useful for the preparation of compounds of the general formula (I) comprises the following steps:

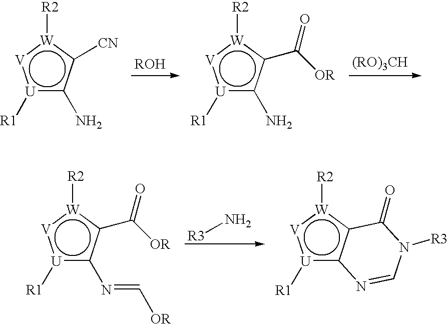

On each occurrence, R is an independently selected organic residue, preferably an alkyl group, in particular a lower alkyl group and most preferably an ethyl group. U, V and W as well as R1, R2 and R3 are defined as above.

Suitable reagents and reaction conditions can be determined by the skilled person. The first step of the above reaction is preferably carried out in the presence of an activating compound, such as an acidic compound, in particular HCl. The second step is preferably carried out in the presence of an activating compound, such as p-TsOH. The reaction is preferably carried out with heating. The third step of the above reaction is preferably carried out in a solvent, in particular ROH, wherein R is defined as above. The reaction is preferably carried out with heating.

A particularly preferred embodiment of the third process is depicted below:

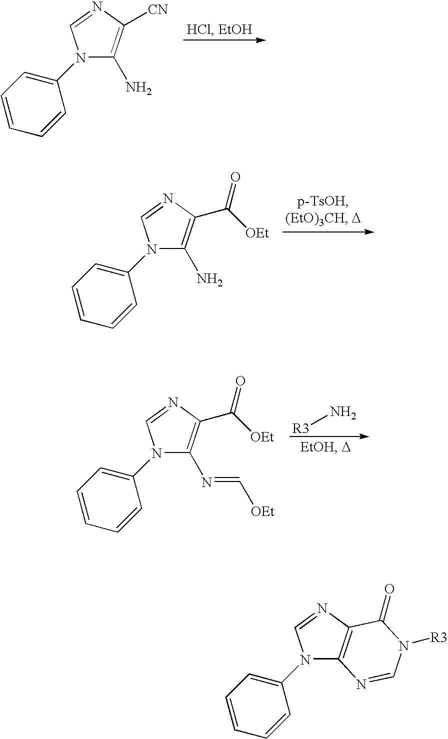

A fourth process which is useful for the preparation of compounds of the general formula (I) comprises the following steps:

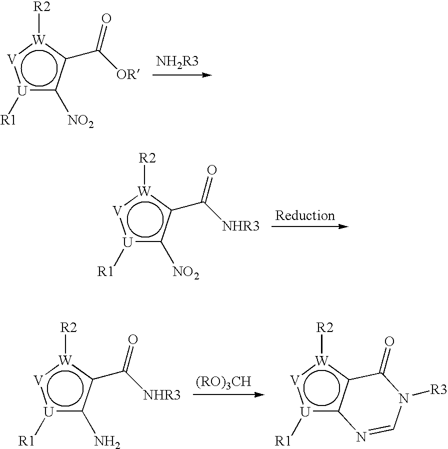

On each occurrence, R is an independently selected organic residue, preferably an alkyl group, in particular a lower alkyl group and most preferably an ethyl group. On each occurrence, R′ is an independently selected organic residue, preferably an alkyl group, in particular a lower alkyl group and most preferably a methyl group. U, V and W as well as R1, R2 and R3 are defined as above.

Suitable reagents and reaction conditions can be determined by the skilled person. The reduction according to the second step of the above reaction is preferably carried out by using SnCl₂ as a reducing agent. The third step of the above reaction is preferably carried out in a solvent, in particular ROH, wherein R is defined as above. The third step is preferably carried out in the presence of an activating compound, such as p-TsOH. The reaction is preferably carried out with heating.

A particularly preferred embodiment of the fourth process is depicted below:

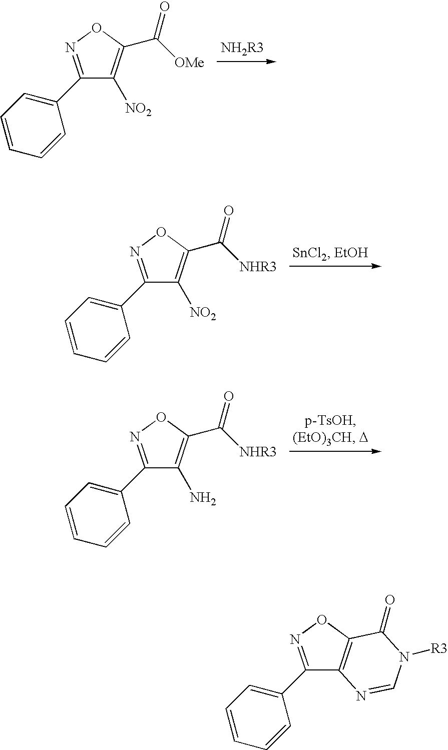

A fifth process which is useful for the preparation of compounds of the general formula (I) wherein W is N and R2′ is alkyl comprises the following steps:

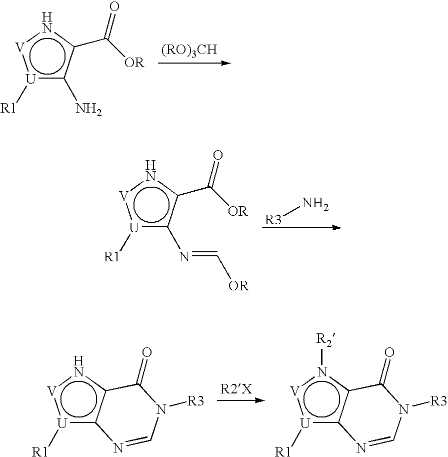

On each occurrence, R is an independently selected organic residue, preferably an alkyl group, in particular a lower alkyl group and most preferably an ethyl group. X is halogen, preferably bromine or iodine, in particular iodine. R2′ is alkyl, preferably lower alkyl, in particular methyl. U and V as well as R1 and R3 are defined as above.

Suitable reagents and reaction conditions can be determined by the skilled person. The first step of the above reaction is preferably carried out in the presence of an activating compound, p-TsOH. The reaction is preferably carried out with heating. The second step of the above reaction is preferably carried out in a solvent, in particular ROH, wherein R is defined as above. The reaction is preferably carried out with heating. The third step of the above reaction is preferably carried out in the presence of an activating compound, in particular a basic compound, such as NaH.

A particularly preferred embodiment of the fifth process is depicted below:

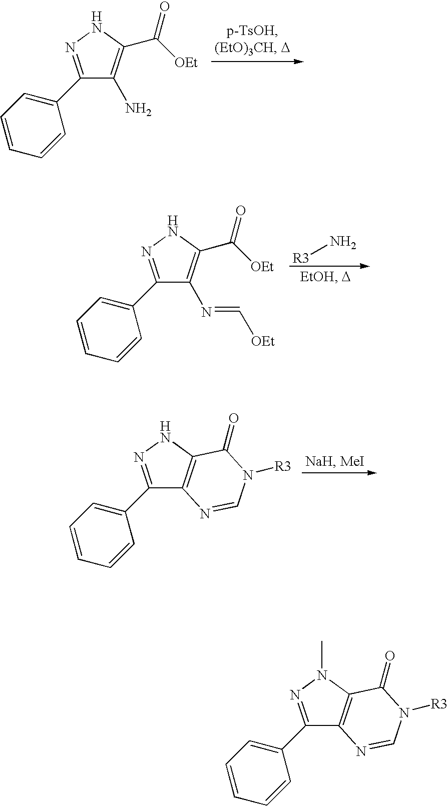

A sixth process which is useful for the preparation of compounds of the general formula (I) comprises the following steps:

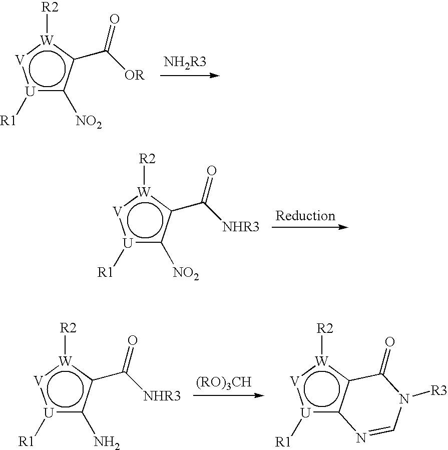

On each occurrence, R is an independently selected organic residue, preferably an alkyl group, in particular a lower alkyl group and most preferably an ethyl group. U, V and W as well as R1, R2 and R3 are defined as above.

Suitable reagents and reaction conditions can be determined by the skilled person. The reduction according to the second step of the above reaction is preferably carried out by using SnCl₂ as a reducing agent. The third step of the above reaction is preferably carried out in a solvent, in particular ROH, wherein R is defined as above. The third step is preferably carried out in the presence of an activating compound, such as p-TsOH. The reaction is preferably carried out with heating.

A particularly preferred embodiment of the sixth process is depicted below:

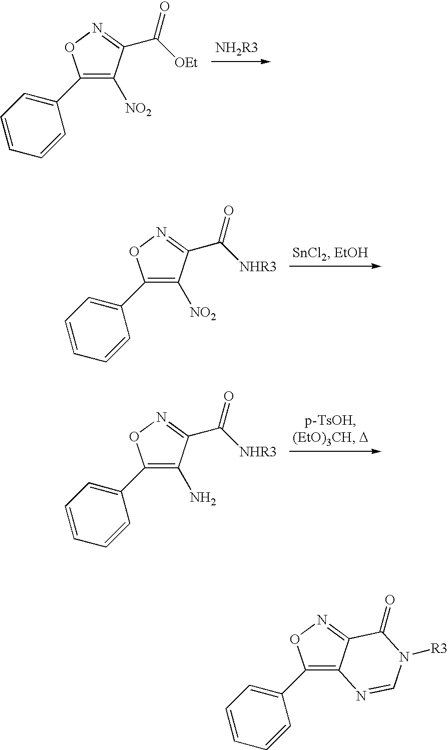

Starting materials are known or obtainable by known methods.

The following considerations apply to the individual reaction steps described above:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/I, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide and Derivate” (Chemistry of carbohydrates: monosaccharides and derivatives), Georg Thieme Verlag, Stuttgart 1974.

b) Acid addition salts may be produced from the free bases in known manner, and vice-versa. Compounds of formula (I) in optically pure form can be obtained from the corresponding racemates according to well-known procedures, e.g. HPLC with chiral matrix. Alternatively, optically pure starting materials can be used.

c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

d) Suitable diluents for carrying out the above-described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethyelene glycol monomethyl ether, diethylene glycol monoethyl ether. Further, mixtures of diluents may be employed. Depending on the starting materials, reaction conditions and auxiliaries, water or diluents constaining water may be suitable. It is also possible to use one starting material as diluent simultaneously.

e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.

f) The reactions are generally carried out under atmospheric pressure. However, it is also possible to carry out the processes according to the invention under elevated or reduced pressure—in general between 0.1 bar and 10 bar.

g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.

h) Work-up is carried out by customary methods (cf. the Preparation Examples).

i) A compound of formula (I) obtained according to the above described processes can be converted into another compound of formula (I) according to conventional methods.

Compounds of formula (I) and their pharmaceutically acceptable acid addition salts, hereinafter referred to as agents of the invention, exhibit valuable pharmacological properties and are therefore useful as pharmaceuticals.

In particular, the agents of the invention exhibit a marked and selective modulating, especially antagonistic, action at human metabotropic glutamate receptors (mGluRs). This can be determined in vitro for example at recombinant human metabotropic glutamate receptors, especially PLC-coupled subtypes thereof such as mGluR5 or mGluR1, using different procedures like, for example, measurement of the inhibition of the agonist induced elevation of intracellular Ca²⁺ concentration in accordance with FF Lin et al., Neuropharm. Vol. 36 (7), pages 917-931 (1997), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1995), L. P. Daggett et al., Neuropharm. Vol. 34, pages 871-886 (1995) and references cited therein. Isolation and expression of human mGluR subtypes are described in U.S. Pat. No. 5,521,297. Selected agents of the invention show IC50 values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR5a or hmGluR1b of about 1 nM to about 50 μM.

The agents of the invention are therefore useful in the prevention, treatment or delay of progression of disorders associated with irregularities of the glutamatergic signal transmission, of the gastro-intestinal and urinary tract and of nervous system disorders mediated full or in part by mGluR group I receptors.

Disorders associated with irregularities of the glutamatergic signal transmission are for example epileptogenesis including neuronal protection after status epilepticus, cerebral ischemias, especially acute ischemias, ischemic diseases of the eye, muscle spasms such as local or general spasticity, skin disorders, obesity disorders and, in particular, convulsions or pain.

Disorders of the gastro-intestinal tract include post-operative ileus, functional gastro-intestinal disorders (FGID) as for example functional dyspepsia (FD), gastro-esophageal reflux disease (GERD), irritable bowel syndrome (IBS), functional bloating, functional diarrhea, chronic constipation, functional disturbancies of the biliary tract as well as other conditions according to Gut 1999; Vol. 45 Suppl. II.

Disorders of the Urinary Tract comprise conditions associated with pain and/or discomfort of the urinary tract and overactive bladder (OAB).

Nervous system disorders mediated fully or in part by mGluR group I receptors are for example acute, traumatic and chronic degenerative processes of the nervous system, such as Parkinson's disease, senile dementia, Alzheimer's disease, Huntington's chorea, amyotrophic lateral sclerosis, multiple sclerosis and fragile X syndrome, substance-related disorders, psychiatric diseases such as schizophrenia, affective and anxiety disorders.

Substance-related disorders include substance abuse, substance dependence and substance withdrawal disorders. Anxiety disorders includes panic disorder, social and specific phobias, anxiety, obsessive compulsive disorder (OCD), post traumatic stress disorder (PTSD) and generalized anxiety disorder (GAD). Affective disorders include depressive (major depression, dysthymia, depressive disorders NOS) and bipolar disorders (bipolar I and II disorders). Other disorders which are mediated fully or in part are pain and itch.

The usefulness of the agents of the invention in the treatment of the above-mentioned disorders can be confirmed in a range of standard tests including those indicated below:

Activity of the agents of the invention in anxiety can be demonstrated in standard models such as the stress-induced hyperthermia in mice [cf. A. Lecci et al., Psychopharmacol. 101, 255-261]. At doses of about 0.1 to about 30 mg/kg p.o., selected agents of the invention reverse the stress-induced hyperthermia.

At doses of about 4 to about 50 mg/kg p.o., selected agents of the invention show reversal of Freund complete adjuvant (FCA) induced hyperalgesia [cf. J. Donnerer et al., Neuroscience 49, 693-698 (1992) and C. J. Woolf, Neuroscience 62, 327-331 (1994)].

For all the above mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.5 to about 100 mg/kg animal body weight. In larger mammals, for example humans, an indicated daily dosage is in the range from about 5 to 1500 mg, preferably about 10 to about 1000 mg of the compound conveniently administered in divided doses up to 4 times a day or in sustained release form.

In accordance with the foregoing, the present invention also provides in a further aspect an agent of the invention for use as a pharmaceutical, e.g. in the treatment of disorders associated with irregularities of the glutamatergic signal transmission, and of nervous system disorders mediated full or in part by mGluR group I receptors, such as mGluR5 or mGluR1.

The invention also provides the use of an agent of the invention, in the treatment of disorders associated with irregularities of the glutamatergic signal transmission, and of nervous system disorders mediated full or in part by mGluR group I receptors, such as mGluR5 or mGluR1.

In a further aspect, the invention provides the use of compounds of formula (I) as modulators of metabotrobic Glutamate Receptors Group I (“mGluR Group I—Modulators”), such as Subtype 5 (“mGluR5—Modulators”) or Subtype 1 (“mGluR1—Modulators”).

Furthermore the invention provides the use of an agent of the invention for the manufacture of a pharmaceutical composition designed for the treatment of disorders associated with irregularities of the glutamatergic signal transmission, and of nervous system disorders mediated full or in part by mGluR group I receptors, such as mGluR5 or mGluR1.

In a further aspect the invention relates to a method of treating disorders mediated full or in part by mGluR group I receptors, such as mGluR5 or mGluR1, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Preferred are the compounds according to the examples.

Further, properly isotope-labeled agents of the invention exhibit valuable properties as histopathological labeling agents, imaging agents and/or biomarkers, hereinafter “markers”, for the selective labeling of group I metabotropic glutamate receptor subtypes (mGlu5 and mGlu1 receptor). More particularly the agents of the invention are useful as markers for labeling the central and peripheral mGluR group 1 receptors in vitro or in vivo. In particular, compounds of the invention which are properly isotopically labeled are useful as ligands to image mGlu5 receptors in vivo or in vitro studies. Suitable radionuclides that may be incorporated in the agents of invention include: 3H, 11C, 13N, 15O, 18F, 123I, 125I, 131I, 75Br, 76Br, 77Br, 82Br, 99mTc and 211At. The choice of radionuclide to be incorporated into compounds of formula (I) will depend on the specific analytical or pharmaceutical application. Therefore, for in vitro labeling of mGluR class I receptors and for competition assays compounds that incorporate 3H, 125I or 77Br would be preferred. For diagnostic and investigating imaging agents (PET or SPECT) compounds that incorporate a radionuclide selected from 11C, 18F, 123I or 76Br are preferred.

The agents of the invention are therefore useful, for instance, for determining the levels of receptor occupancy of a drug acting at mGluR group I receptors, or diagnostic purposes for diseases resulting from an imbalance or dysfunction of mGluR group I receptors, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving mGluR group I receptors in vivo and in vitro comprising an agent of the invention.

In still a further aspect, the present invention provides a method for labeling brain and peripheral nervous system structures involving mGluR group I receptors in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following non-limiting Examples illustrate the invention. A list of Abbreviations used is given below.
BOC tert-butoxycarbonyl
n-BuLi n-butyl lithium
DCM dichloromethane
DMF N,N′-dimethylformamide
EDC 1-ethyl-3-[3-(dimethylamino)propyl]-carbodiimide hydrochloride
EtOAc ethylacetate
h hour(s)
HCl hydrochloric acid
HOBt hydroxybenzotriazole
HPLC high performance liquid chromatography
min minute(s)
Mp melting point
MS mass spectroscopy
MTBE methyl-tert.-butylether
Rf retention factor (Thin Layer Chromatography)
rt room temperature
TFA trifluoroacetic acid
THF tetrahydrofuran
UPLC ultra performance liquid chromatography

HPLC Specificity

System A: Agilent 1100 Series, LC-MSD and a Macherin Nagel Nucleosil C-18HD 4×70 mm 3 μm. Column running a gradient Water+0.05% TFA/Acetonitrile+0.05% TFA from 80/20 to 0/100 over 6′-0/100 over 1.5′-0/100 to 80/20 over 0.5′ with a flux of 1.0 ml/min, 35° C.

System B: Agilent 1100 Series, LC-MSD and a Agilent Zorbax SB-C18 3×30 mm 1.8 μm Column running a gradient Water+0.05% TFA/Acetonitrile+0.05% TFA from 70/30 to 0/100 over 3.25′-0/100 over 0.75′-0/100 to 70/30 over 0.25′ with a flux of 0.7 ml/min, 35° C.

System C: Agilent 1100 Series, LC-MSD and a Agilent Zorbax SB-C18 3×30 mm 1.8 μm Column running a gradient Water+0.05% TFA/Acetonitrile+0.05% TFA from 60/40 to 0/100 over 3.25′-0/100 over 0.75′-0/100 to 60/40 over 0.25′ with a flux of 0.7 ml/min, 35° C.

System D: Agilent 1100 Series, LC-MSD and a Agilent Zorbax SB-C18 3×30 mm 1.8 μm Column running a gradient Water+0.05% TFA/Acetonitrile+0.05% TFA from 90/10 to 0/100 over 3.25′-0/100 over 0.75′-0/100 to 90/10 over 0.25′ with a flux of 0.7 ml/min, 35° C.

System E: UPLC (Waters Acquity; column type Acquity UPLC BEH C18, 2.1×50 mm, particle size 1.7 μm, gradient: 5-100% acetonitrile (0.1% TFA)/H₂O (0.1% TFA) over 2 min, 100% acetonitrile (0.1% TFA) for 0.5 min): single peak at the indicated retention time Rt (minutes).

### EEXAMPLE 1

### 5-Cyclohexyl-1-pyridin-4-yl-1,5-dihydro-pyrazolo[3,4-d]pyrimidin-4-one

A solution of 5-ethoxymethyleneamino-1-pyridin-4-yl-1H-pyrazole-4-carboxylic acid ethyl ester (0.688 g, 2.39 mmol) and cyclohexylamine (0.82 ml, 7.16 mmol, 3 eq) in EtOH (5 ml) were stirred at 78° C. for 12 h. After cooling the solution to room temperature, the solvent was evaporated under reduced pressure, the residue taken up in DCM (5 ml) and washed with 0.1 M HCl and aqueous solution of sodium bicarbonate. The organic layer was dried over Na₂SO₄ and the solvent evaporated to afford a crude product that was purified by chromatography on silica gel to afford 36 mg (5%) of the desired product.

MS (LC/MS): 296.2 [M+H]

HPLC Rt: 2.97 min (gradient elution) System A

The starting material was prepared as described hereafter:

### i) 5-Ethoxymethyleneamino-1-pyridin-4-yl-1H-pyrazole-4-carboxylic acid ethyl ester

A solution of 5-amino-1-pyridin-4-yl-1H-pyrazole-4-carboxylic acid ethyl ester (700 mg, 2.95 mmol), p-TsOH (13.3 mg, 0.0737 mmol, 0.02 eq) and triethylorthoformate (3.01 ml, 17.7 mmol, 6 eq) was stirred at 80° C. for 2 h. After removing excess of triethylorthoformate by distillation, hexanes (5 ml) was added to the remaining mixture to induce crystallization of pure product (688 mg, 81%).

### ii) 5-Amino-1-pyridin-4-yl-1H-pyrazole-4-carboxylic acid ethyl ester

To a solution of 2-cyano-3-ethoxy-acrylic acid ethyl ester (1.55 g, 9.16 mmol) and triethlamine (1.4 ml, 10.1 mmol, 1.1 eq) in EtOH (20 ml) was added pyridin-4-yl-hydrazine (1.0 g, 9.16 mmol). After stirring the resulting yellow suspension at 25° C. for 30 min and at 78° C. for 2 h the mixture was allowed to reach room temperature and the solvent was removed subsequently. Dilution of the mixture with EtOAc, washing with sodium bicarbonate solution and brine, drying of the organic phase with Na₂SO₄, filtration and evaporation of the solvent afforded a desired product (1.83 g, 86%) which could be used without further purification.

Following the same procedures, the following compounds can be obtained:

### EXAMPLE 2

### 5-Cyclohexyl-1-m-tolyl-1,5-dihydro-pyrazolo[3,4-d]pyrimidin-4-one

MS (LC/MS): 309.2 [M+H]

HPLC Rt: 5.80 min (gradient elution) System A

TLC Rf: 0.76 (EtOAc/hexane 1:1)

### EXAMPLE 3

### 5-Cyclohexyl-1-pyridin-3-yl-1,5-dihydro-pyrazolo[3,4-d]pyrimidin-4-one

MS (LC/MS): 296.2 [M+H]

HPLC Rt: 1.32 min (gradient elution) System B

TLC Rf: 0.28 (EtOAc/hexane 1:1)

### EXAMPLE 4

### 1-(2-Chloro-phenyl)-5-cyclohexyl-1,5-dihydro-pyrazolo[3,4-d]pyrimidin-4-one

MS (LC/MS): 329 [M+H]

HPLC Rt: 5.03 min (gradient elution) System A

TLC Rf: 0.20 (EtOAc/hexane 1:1)

### EXAMPLE 5

### 5-Cyclohexyl-1-pyridin-2-yl-1,5-dihydro-pyrazolo[3,4-d]pyrimidin-4-one

MS (LC/MS): 296.2 [M+H]

HPLC Rt: 3.93 min (gradient elution) System A

TLC Rf: 0.18 (EtOAc/hexane 1:1)

### EXAMPLE 6

### 1-(3-Chloro-phenyl)-5-cyclohexyl-1,5-dihydro-pyrazolo[3,4-d]pyrimidin-4-one

MS (LC/MS): 329.0 [M+H]

HPLC Rt: 6.22 min (gradient elution) System A

### EXAMPLE 7

### 5-Cyclohexyl-1-o-tolyl-1,5-dihydro-pyrazolo[3,4-d]pyrimidin-4-one

MS (LC/MS): 309.2 [M+H]

HPLC Rt: 5.10 min (gradient elution) System A

TLC Rf: 0.49 (EtOAc/hexane 1:2)

### EXAMPLE 8

### 5-Cyclohexyl-1-p-tolyl-1,5-dihydro-pyrazolo[3,4-d]pyrimidin-4-one

MS (LC/MS): 309.2 [M+H]

HPLC Rt: 5.82 min (gradient elution) System A

TLC Rf: 0.55 (EtOAc/hexane 1:2)

### EAXMPLE 10

### 1-(4-Chloro-phenyl)-5-cyclohexyl-1,5-dihydro-pyrazolo[3,4-d]pyrimidin-4-one

MS (LC/MS): 329.0 [M+H]

HPLC Rt: 5.23 min (gradient elution) System A

TLC Rf: 0.36 (EtOAc/hexane 1:1)

### EXAMPLE 11

### 5-Cycloheptyl-3-methyl-1-phenyl-1,5-dihydro-pyrazolo[3,4-d]pyrimidin-4-one

MS (LC/MS): 323.2 [M+H]

UPLC Rt: 1.826 min (System E)

TLC Rf: 0.57 (EtOAc/hexane 3:7)

### EXAMPLE 12

### 5-Cycloheptyl-3-methoxy-1-phenyl-1,5-dihydro-pyrazolo[3,4-d]pyrimidin-4-one

MS (LC/MS): 339.2 [M+H]

UPLC Rt: 1.792 min (System E) TLC Rf: 0.45 (EtOAc/hexane 3:7)

The starting material was prepared as described hereafter:

### i) 5-Amino-3-methoxy-1-phenyl-1H-pyrazole-4-carboxylic acid ethyl ester

A solution 5-amino-3-methoxy-1-phenyl-1H-pyrazole-4-carbonitrile (237 mg, 1.11 mmol) and conc. sulfuric acid (0.5 ml) in ethanol (15 ml) was stirred at 80° C. for 16 h. Then, more conc. sulfuric acid (1 ml) was added and the mixture stirred at 80° C. for 7 h; then a third portion of conc. sulfuric acid was added and stirring continued at 80° C. After a total reaction time of 40 h, the ethanol was removed under reduced pressure, the residue cooled to 0° C. and neutralized carefully with NaHCO₃-soln. (5%). The aqueous phase was extracted with DCM (3×), the combined organic phases dried (Na₂SO₄) and concentrated. Purification by chromatography on silica gel yielded the desired product (49 mg, 17%).

### EXAMPLE 13

### 5-Cycloheptyl-3-ethyl-1-phenyl-1,5-dihydro-pyrazolo[3,4-d]pyrimidin-4-one

MS (LC/MS): 337.2 [M+H]

UPLC Rt: 1.992 min (System E)

TLC Rf: 0.53 (EtOAc/hexane 3:7)

### EXAMPLE 14

### 5-Cycloheptyl-1-phenyl-3-propyl-1,5-dihydro-pyrazolo[3,4-d]pyrimidin-4-one

MS (LC/MS): 351.2 [M+H]

UPLC Rt: 2.103 min (System E)

TLC Rf: 0.74 (EtOAc/hexane 3:7)

### EXAMPLE 15

### 5-Cycloheptyl-1,3-diphenyl-1,5-dihydro-pyrazolo[3,4-d]pyrimidin-4-one

MS (LC/MS): 385.2 [M+H]

UPLC Rt: 2.165 min (System E)

TLC Rf: 0.44 (EtOAc/hexane 2:8)

### EXAMPLE 16

### 5-Cycloheptyl-3-methyl-1-phenyl-1,5-dihydro-pyrazolo[3,4-d]pyrimidin-4-one

To a solution of chloro-trimethyl-silane (44 mg, 0.40 mmol, 4 eq) and potassium iodide (67 mg, 0.40 mmol, 4 eq) in acetonitrile (2 ml) was added 5-cycloheptyl-3-methoxy-1-phenyl-1,5-dihydro-pyrazolo[3,4-d]pyrimidin-4-one (34 mg, 0.10 mmol) dissolved in acetonitrile (1 ml). The resulting mixture was stirred at 80° C. for 90 minutes. After cooling to room temperature, the reaction was quenched with Na₂S₂O₃-soln. and the solvents were removed under reduced pressure. The residue was dissolved in H₂O, extracted with DCM (3×), the combined organic phases dried (Na₂SO₄) and concentrated. Purification by chromatography on silica gel yielded the desired product (23 mg, 71%).

MS (LC/MS): 325.2 [M+H]

UPLC Rt: 1.503 min (System E)

TLC Rf: 0.57 (MeOH/DCM 5:95)

Following the procedures described in example 1, the following compounds can be obtained

### EXAMPLE 17

### 5-Cyclooctyl-1-phenyl-1,5-dihydro-pyrazolo[3,4-d]pyrimidin-4-one

MS (LC/MS): 323.2 [M+H]

HPLC Rt: 6.08 min (gradient elution) System A

TLC Rf: 0.38 (EtOAc/hexane 1:4)

### EXAMPLE 18

### 5-Adamantan-2-yl-1-phenyl-1,5-dihydro-pyrazolo[3,4-d]pyrimidin-4-one

MS (LC/MS): 347.2 [M+H]

HPLC Rt: 3.57 min (gradient elution) System C

TLC Rf: 0.25 (EtOAc/hexane 1:4)

### EXAMPLE 19

### 1-Phenyl-5-piperidin-1-yl-1,5-dihydro-pyrazolo[3,4-d]pyrimidin-4-one

MS (LC/MS): 296.2 [M+H]

HPLC Rt: 3.28 min (gradient elution) System B

TLC Rf: 0.53 (EtOAc/hexane 1:1)

### EXAMPLE 20

### 5-Bicyclo[3.2.1]oct-3-yl-1-phenyl-1,5-dihydro-pyrazolo[3,4-d]pyrimidin-4-one

MS (LC/MS): 321.2 [M+H]

HPLC Rt: 3.55 min (gradient elution) System B

TLC Rf: 0.63 (EtOAc/hexane 1:1)

### EXAMPLE 21

### 5-Azepan-1-yl-1-phenyl-1,5-dihydro-pyrazolo[3,4-d]pyrimidin-4-one

MS (LC/MS): 310.2 [M+H]

HPLC Rt: 3.60 min (gradient elution) System B

TLC Rf: 0.65 (EtOAc/hexane 1:1)

### EXAMPLE 22

### 5-Cyclopentyl-1-phenyl-1,5-dihydro-pyrazolo[3,4-d]pyrimidin-4-one

MS (LC/MS): 281.2 [M+H]

HPLC Rt: 5.00 min (gradient elution) System A

TLC Rf: 0.46 (EtOAc/hexane 1:1)

### EXAMPLE 23

### 5-(8-Methyl-8-aza-bicyclo[3.2.1]oct-3-yl)-1-phenyl-1,5-dihydro-pyrazolo[3,4-d]pyrimidin-4-one

MS (LC/MS): 336.2 [M+H]

HPLC Rt: 2.48 min (gradient elution) System D

TLC Rf: 0.65 (DCM/MeOH 95:5+triethylamine)

### EXAMPLE 24

### 5-(3-Chloro-phenyl)-1-phenyl-1,5-dihydro-pyrazolo[3,4-d]pyrimidin-4-one

MS (LC/MS): 323.0 [M+H]

HPLC Rt: 5.19 min (gradient elution) System A

TLC Rf: 0.63 (DCM/MeOH 95:5)

### EXAMPLE 25

### 6-Cycloheptyl-3-phenyl-3,6-dihydro-[1,2,3]triazolo[4,5-d]pyrimidin-7-one

MS (LC/MS): 310.2 [M+H]

UPLC Rt: 1.589 min (System E)

### EXAMPLE 26

### 1-Cycloheptyl-9-phenyl-1,9-dihydro-purin-6-one

MS (LC/MS): 309.2 [M+H]

HPLC Rt: 3.47 min (gradient elution) System D

TLC Rf: 0.30 (EtOAc/hexane 1:1)

The starting material was prepared as described hereafter:

### i) 5-Amino-1-phenyl-1H-imidazole-4-carboxylic acid ethyl ester

A solution of 5-amino-1-phenyl-1H-imidazole-4-carbonitrile (1.55 g, 8.42 mmol), water (4 ml) and concentrated ethanolic hydrochloric acid (140 ml) was stirred at 78° C. for 3 d. After allowing the reaction mixture to reach room temperature, the solution was neutralized to pH=7 by addition of sodium bicarbonate and extracted twice with EtOAc. Combined organic phases were dried over Na₂SO₄, the solvent was evaporated under reduced pressure and the residue purified by chromatography over silica gel to afford the desired product (0.35 g, 18%).

### EXAMPLE 27

### 6-Cycloheptyl-3-phenyl-6H-isoxazolo[4,5-d]pyrimidin-7-one

A solution of 4-amino-3-phenyl-isoxazole-5-carboxylic acid cycloheptylamide (241 mg, 0.81 mmol) and p-TsOH (8 mg, 0.04 mmol, 0.05 eq) in triethylorthoformate (716 mg, 4.83 mmol, 6 eq) was stirred under nitrogen atmosphere at 130° C. for 16 h. After cooling the solution to room temperature, the crude mixture was purified by chromatography on silica gel to give 122 mg (49%) of the desired product.

UPLC Rt: 1.414 min (System E)

TLC Rf: 0.22 (MeOH/DCM 5:95)

The starting material was prepared as described hereafter:

### i) 4-Amino-3-phenyl-isoxazole-5-carboxylic acid cycloheptylamide

A solution of 4-nitro-3-phenyl-isoxazole-5-carboxylic acid cycloheptylamide (500 mg, 1.52 mmol) and dry SnCl₂ (1.44 g, 7.59 mmol, 5 eq) in ethanol (5 ml) was stirred at 50° C. for 1 h and then at 75° C. for 3 h. The mixture was dissolved in DCM and washed with NaOH (1M), the aqueous phase extracted with DCM, the combined organic phases dried (Na₂SO₄) and concentrated. This afforded the desired product (241 mg, 48%) which could be used without further purification.

MS (LC/MS): 300.2 [M+H]

UPLC Rt: 1.042 min (System E)

### ii) 4-Nitro-3-phenyl-isoxazole-5-carboxylic acid cycloheptylamide

4-Nitro-3-phenyl-isoxazole-5-carboxylic acid methyl ester (2.0 g, 8.06 mmol) was dissolved in cycloheptylamine (1.54 ml). After stirring the resulting yellow solution at 75° C. for 2 h another 0.5 ml of cycloheptylamine were added. After a total reaction time of 2 h, the mixture was allowed to reach room temperature and then taken up in DCM, washed with H₂O, the combined organic phases dried (Na₂SO₄) and concentrated. Purification by chromatography on silica gel gave 1.01 g (41%) of the desired product.

MS (LC/MS): 330.2 [M+H]

UPLC Rt: 1.679 min (System E)

Following the same procedures, the following compound can be obtained:

### EXAMPLE 29

### 6-Cycloheptyl-3-phenyl-6H-isoxazolo[4,3-d]pyrimidin-7-one

MS (LC/MS): 310.2 [M+H]

UPLC Rt: 1.480 min (System E)

TLC Rf: 0.22 (MeOH/DCM 5:95)

Following the same procedures as described in example 1, the following compounds can be obtained

### EXAMPLE 28

### 6-Cycloheptyl-3-phenyl-1,6-dihydro-pyrazolo[4,3-d]pyrimidin-7-one

MS (LC/MS): 309.2 [M+H]

HPLC Rt: 3.70 min (gradient elution) System D

TLC Rf: 0.60 (EtOAc/hexane 1:1)

### EXAMPLE 30

### 1-Cyclooctyl-9-phenyl-1,9-dihydro-purin-6-one

MS (LC/MS): 323.2 [M+H]

HPLC Rt: 3.62 min (gradient elution) System D

TLC Rf: 0.36 (EtOAc/hexane 1:1)

### EXAMPLE 31

### 6-Cyclooctyl-3-phenyl-1,6-dihydro-pyrazolo[4,3-d]pyrimidin-7-one

MS (LC/MS): 323.2 [M+H]

HPLC Rt: 3.85 min (gradient elution) System D

TLC Rf: 0.11 (EtOAc/hexane 1:1)

### EXAMPLE 32

### 6-Cyclohexyl-3-phenyl-1,6-dihydro-pyrazolo[4,3-d]pyrimidin-7-one

MS (LC/MS): 295.2 [M+H]

HPLC Rt: 3.53 min (gradient elution) System D

TLC Rf: 0.59 (EtOAc/cyclohexane 1:1)

### EXAMPLE 33

### 6-Cyclooctyl-1-methyl-3-phenyl-1,6-dihydro-pyrazolo[4,3-d]pyrimidin-7-one

To a suspension of 6-cyclooctyl-3-phenyl-1,6-dihydro-pyrazolo[4,3-d]pyrimidin-7-one (50 mg, 0.155 mmol) in DMF (1 ml) was added sodium hydride (60% in mineral oil, 7.8 mg, 0.195 mmol, 1.26 eq). After stirring the solution for 15 min, methyl iodide (12.6 μL, 0.202 mmol, 1.3 eq) was added. After stirring for additional 15 min, the reaction mixture was quenched by addition of water (1.5 ml) and extracted twice with ethyl acetate. Combined organic layers were washed with water, dried over sodium sulfate and the solvent evaporated under reduced pressure to afford pure product (43 mg, 82%) after purification by chromatography over silica gel.

MS (LC/MS): 337.2 [M+H]

HPLC Rt: 4.03 min (gradient elution) System B

TLC Rf: 0.50 (EtOAc/hexane 1:1)

1. A compound of formula (I) or a tautomeric form thereof 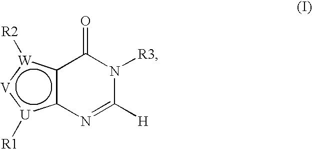
wherein
U represents C or N;
V represents CH, N, or O;
W represents C, N, or O;
R1 represents an optionally substituted aryl or an optionally substituted heteroaryl group;
R2 if present is selected from the group consisting of H, alkyl, aryl, hydroxy or alkoxy;
R3 is selected from the group consisting of optionally substituted cycloalkyl, optionally substituted heterocycloalkyl or 3-Cl-phenyl;
in free base or acid addition salt form,
with the proviso, that the compound of the formula 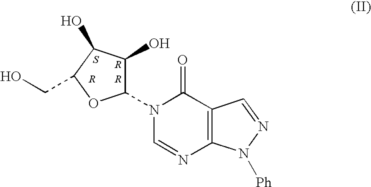
is excluded.

2. A compound according to claim 1 wherein U—V—W together represent N—N—C; N—CH—N; C—N—N; N—N—N; C—N—O; or C—O—N.

3. A compound according to claim 1, wherein R1 represents phenyl or a six-membered heteroaryl ring containing one N.

4. A compound according to claim 1, wherein the aryl or heteroaryl group which is represented by R1, is unsubstituted or carries 1 to 3 substituents.

5. A compound according to claim 4 wherein substituents of the group represented by R1 are selected among halogen and lower alkyl.

6. A compound according to claim 1 wherein R2 is absent or is selected from the group consisting of H, lower alkyl, phenyl, hydroxy or lower alkoxy.

7. A compound according to claim 1 wherein R3 is selected from the group consisting of an optionally substituted C5-C8 monocyclic alkyl group, an optionally substituted bicyclic alkyl group containing 7 to 12 carbon atoms in the bicyclic moiety, an optionally substituted heterocyclic group derivable from an optionally substituted C5-C8 monocyclic alkyl group or an optionally substituted bicyclic alkyl group containing 7 to 12 carbon atoms in the bicyclic moiety by replacing a CH group in one of the rings by a nitrogen atom and 3-chloro phenyl.

8. A compound according to claim 4 wherein R1-U is selected from the group consisting of

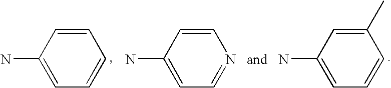

9. A compound according to claim 1 wherein V is N.

10. A compound according to claim 6 wherein R2-W is selected from the group consisting of CH and N.

11. A compound according to claim 7 wherein R3 is selected from the group consisting of cyclohexyl, cycloheptyl, cyclooctyl,

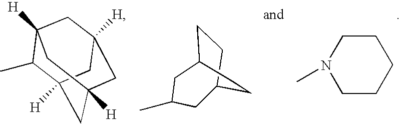

12. A pharmaceutical composition comprising a compound according to claim 1 and a pharmaceutical carrier or diluent.

13-18. (canceled)

19. A method of treating, preventing or delaying the progression of a disorder associated with irregularities of the glutamatergic signal transmission of the gastrointestinal or urinary tract or a nervous system disorders mediated full or in part by mGluR group I receptors, comprising administering to a subject a compound represented of formula (I) or a tautomeric form thereof 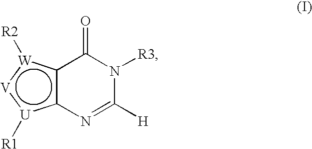
wherein
U represents C or N;
V represents CH, N, or O;
W represents C, N, or O;
R1 represents an optionally substituted aryl or an optionally substituted heteroaryl group;
R2 if present is selected from the group consisting of H, alkyl, aryl, hydroxy or alkoxy;
R3 is selected from the group consisting of optionally substituted cycloalkyl, optionally substituted heterocycloalkyl or 3-Cl-phenyl;
in free base or acid addition salt form.

20. The method according to claim 19, wherein the disorder of the nervous system mediated full or in part by mGluR group I receptors is selected from the group consisting of acute, Parkinson's disease, senile dementia, Alzheimer's disease, Huntington's chorea, amyotrophic lateral sclerosis, multiple sclerosis, fragile X syndrome, schizophrenia, affective and anxiety disorders, substance abuse, substance dependence, substance withdrawal disorders, panic disorder, social and specific phobias, anxiety, obsessive compulsive disorder (OCD), post traumatic stress disorder (PTSD), generalized anxiety disorder (GAD), major depression, dysthymia, depressive disorders NOS and bipolar disorders.

21. The method according to claim 19, wherein the disorders of the urinary tract comprise conditions associated with pain and/or discomfort of the urinary tract, overactive bladder (OAB), or a combination thereof.

22. The method according to claim 19, wherein the disorder of the gastro-intestinal tract is selected from the group consisting of post-operative ileus, functional dyspepsia (FD), gastro-esophageal reflux disease (GERD), irritable bowel syndrome (IBS), functional bloating, functional diarrhea, chronic constipation, and functional disturbancies of the biliary tract.

23. The method according to claim 19, wherein the disorder associated with irregularities of the glutamatergic signal transmission is selected from the group consisting of epileptogenesis, cerebral ischemias, muscle spasms, skin disorders, obesity disorders, convulsions and pain.
